(19) United States
(12) Patent Application Publication
(10) Pub. No.: US 2022/0071924 A1
(43) Pub. Date: Mar. 10, 2022

(54) TREATMENT OF OCULAR DISEASES WITH OPHTHALMIC TAPINAROF COMPOSITIONS

(71) Applicant: Sol-Gel Technologies Ltd., Ness Ziona (IL)

(72) Inventors: Moshe ARKIN, Kfar Shmaryahu (IL); Marcel ZIGHELBOIM, Kiryat Motzkin (IL)

(73) Assignee: Sol-Gel Technologies Ltd., Ness Ziona (IL)

(21) Appl. No.: 17/527,582

(22) Filed: Nov. 16, 2021

Related U.S. Application Data
(63) Continuation-in-part of application No. 16/994,940, filed on Aug. 17, 2020.
Application No. 16/994,940 is a continuation-in-part of application No. 16/935,452, filed on Jul. 22, 2020.
Application No. 16/935,452 is a continuation-in-part of application No. 16/929,400, filed on Jul. 15, 2020.
Application No. 16/929,400 is a continuation-in-part of application No. PCT/IL2020/050677, filed on Jun. 17, 2020.
Application No. PCT/IL2020/050677 is a continuation-in-part of application No. 17/440,778.
(60) Provisional application No. 62/862,141, filed on Jun. 17, 2019.
(60) Provisional application No. 62/820,498, filed on Mar. 19, 2019.

(51) Int. Cl. A61K 31/05; A61P 33/00; A61K 9/00

(52) U.S. Cl. CPC A61K 31/05; A61K 9/0014; A61P 33/00

(57) ABSTRACT

The present invention relates to the treatment of an ocular inflammatory disease or an ocular degeneration disease by ophthalmic administration of a composition comprising tapinarof and optionally at least one additional active agent. Specifically, the ocular inflammatory disease is blepharitis. This invention provides a treatment of blepharitis and Demodex mites.

### CROSS REFERENCE TO RELATED APPLICATIONS

This application is a Continuation-in-Part from U.S. patent application Ser. No. 16/994,940, filed Aug. 17, 2020, which is a Continuation-in-Part of U.S. patent application Ser. No. 16/935,452, filed on Jul. 22, 2020, which is a Continuation-in-Part of U.S. patent application Ser. No. 16/929,400, filed on Jul. 15, 2020, which is a Continuation-in-Part of PCT International Application No. PCT/IL2020/050677 filed on Jun. 17, 2020, which claims the benefit of U.S. Ser. No. 62/862,141, filed on Jun. 17, 2019;

This application is a Continuation-in-Part from U.S. patent application Ser. No. 17/440,778, filed Sep. 19, 2021 which is a National Phase Application of PCT International Application No. PCT/IL2020/050334, International Filing Date Mar. 19, 2020, claiming the benefit of U.S. Patent Application No. 62/820,498, filed Mar. 19, 2019, which are hereby incorporated in their entirety herein by reference.

### FIELD OF THE INVENTION

The present invention, in some embodiments thereof, relates to treatment of an ocular inflammatory disease or an ocular degeneration disease by topical administration to a subject in need thereof of a composition comprising tapinarof and optionally at least one additional active agent. The composition of this invention is useful for the treatment, prevention or amelioration of ocular inflammatory diseases or of ocular degeneration disease and their symptoms.

### BACKGROUND OF THE INVENTION

Ocular inflammatory diseases and/or ocular degeneration diseases include uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), orbital myositis, and combinations thereof. Ocular autoimmune diseases have been described in a recent article by Bose T. et al, “Dry eye disease and uveitis”, Autoimmune Reviews, vol. 15, issue 12, December 2016, pp. 1181-1192.

While DED affects the ocular surface, uveitis involves inflammation of the inner eye.

Uveitis is the inflammation of the uvea, the pigmented layer between the inner retina and the outer fibrous layer composed of the sclera and cornea. According to the site of the inflammation, there are several types of uveitis: anterior uveitis, interior uveitis, posterior uveitis and pan-uveitis (inflammation of all the uvea layers). Uveitis affects literally millions of people around the globe, with women dramatically over represented, particularly those women who have entered menopause. It has the potential for serious ocular consequences, beginning with the formation of dry spots on the cornea, progressing to epithelial defects or “abrasions” which resist healing, and then in some instances causing ulceration of the cornea, sometimes with perforation. Present medication of uveitis includes topical steroids (prednisolone acetate), injectable triamcinolone acetate, topical atropine or homatropine and methotrexate.

Dry eye disease (DED), also known as dry eye syndrome (DES) or keratoconjunctivitis sicca (KCS), affects up to 70% of older people. Medication of DED includes mild topical steroids (like prednisolone), lubricating tear ointments, immune-suppressants (like ciclosporin) and lifitegrast, Lifitegrast (Xiidra) is a new immune mediator drop for management of dry eye shown to be effective. (“Dry Eye”—The Ocular Immunology and Uveitis Foundation—www.uveitis.org). Some of the above treatments exhibit unwanted side-effects.

Blepharitis is an inflammation of the eyelids in which they become red, irritated and itchy and dandruff-like scales form on the eyelashes. Blepharitis is one of the most common ocular conditions and affects people of all ages. Blepharitis is typically caused by bacterial infection or blockage of the meibomian oil glands. Diseases and conditions that may lead to blepharitis include: rosacea, herpes, simplex dermatitis, varicella-zoster dermatitis, molluscum contagiosum, allergic dermatitis, contact dermatitis, demodicosis (Demodex), and parasitic infections (e.g., Demodex and Phthiriasis palpebrarum).

Demodex mites are known inhabitants of human eyelash follicles and glands, and appear at an increasing incidence with age. Demodex folliculorum and Demodex brevis, are implicated in anterior blepharitis and posterior blepharitis respectively. Over time, the idea of Demodex as a major etiology for blepharitis has become increasingly popular. Demodex are thought to cause blepharitis through multiple mechanisms including: epithelial hyperplasia and hyperkeratosis, bacterial vectors, direct obstruction of meibomian glands, and immunological responses secondary to mite carcasses and excreta.

The Demodex mite is an ectoparasite that can reside in hair follicles and sebaceous glands. Demodex mites can infest the eyelids, eyelashes, and glands of surrounding ocular tissue (e.g., meibomian glands).

Currently, no widely accepted gold-standard exists for the treatment of Demodex blepharitis. Treatments can include lid hygiene, hypochlorous acid, sulfur 1% ointment, metronidazole 2% ointment, ivermectin 1% cream, Cilclar 1.9%+Oxide Mercury+Ether, systemic ivermectin, systemic metronidazole, tea tree oil, intense pulsed light therapy, microblepheroexfoliation with and without terpinen-4-ol. Tea tree oil and its principally active ingredient terpinen-4-ol have been gaining popularity as the treatment of choice for demodicosis. However, well established side effects including contact dermatitis, ocular irritation, and allergic reactions can form a therapeutic barrier to Demodex eradication. In vitro data has demonstrated that even sub therapeutic concentrations of terpinen-4-ol can be toxic to meibomian gland epithelial cells. This underscores the need for further research into new treatments for Demodex blepharitis.

Blepharitis can be divided into anterior and posterior according to anatomic location: a) Anterior blepharitis occurs at the outside front edge of the eyelid where the eyelashes attach; b) Posterior blepharitis affects the inner edge of the eyelid that touches the eyeball. The parasite Demodex folliculorum (D. folliculorum) causes blepharitis when the parasite is present in excessive numbers within the dermis of the eyelids. The life span of Demodex mites ranges 14-18 days. The parasites (both adult and eggs) live on the hair follicle, inhabiting the sebaceous and apocrine gland of the human lid. Direct contact allows this pathogen to spread. Thyroid eye disease (TED) is a degenerative disease that manifests with detrimental tissue remodeling, myofibroblast accumulation, and scarring in the orbit of affected individuals. Currently, there are no effective therapies for TED that target or prevent the excessive tissue remodeling caused by myofibroblast formation and activation. Thyroid eye disease (TED), previously called thyroid associated ophthalmopathy or Graves ophthalmopathy, is a serious condition that affects up to 50% of people with Graves disease. Current treatments for TED include corticosteroids, external beam radiation, and invasive surgery, which can cause adverse effects, including edema, radiation exposure, and postsurgical morbidity. These treatments are aimed at the consequences of disease rather than targeting key effector cells, such as the myofibroblast.

Ocular hyperemia, or red eye, referred herein as “hyperemia” is a condition caused by abnormalities associated with the blood vessels at affected locations, often on the eye or skin. Excess vessel growth and increased vascularity is one of the major causes. In addition, abnormal vessel dilation or leakage often lead to hyperemia. Many diseases and conditions, such as inflammation, can induce hyperemia.

Seborrheic dermatitis is a subacute or chronic inflammatory disorder confined to the sebaceous gland-rich skin of the head, the trunk, and occasionally, the intertriginous areas. Seborrheic dermatitis has a predilection for the hairy regions of the skin, where sebaceous glands are numerous. These regions are the scalp, eyebrows, eyelids, nasolabial creases, ears, chest, intertriginous areas, axilla, groin, buttocks, and inframammary folds. The rash is bilateral and symmetrically distributed. In its mildest form, dandruff, one sees fine, white scale without erythema. This invention is directed to Seborrheic dermatitis of the eyebrows and eyelids.

The mainstay of treatment for dry eye syndrome through the years has been replacement of fluid through the use of artificial tears. And while this is an important approach to the treatment of dry eye, it is by no means the only or the most important approach. The use of anti-inflammatory and immunomodulatory agents by topical application also plays a role in many patients' dry eye care, since inflammation of the lacrimal gland has been shown to be present and to be hindering the normal function of that tear-producing gland in a significant proportion of patients with dry eye disease. Hence, topical Alrex (loteprednol etabonate, a weak corticosteroid) and topical Restasis (containing cyclosporin) have been shown to be beneficial in such patients.

Macular degeneration, especially age-related macular degeneration (AMD or ARMD), a disease in which inflammation plays a clear role, is the leading cause of blindness. With ageing populations in many countries, more than 20% might have the disorder. Advanced age-related macular degeneration, including neovascular age-related macular degeneration (wet) and geographic atrophy (late dry), is associated with substantial, progressive visual impairment. The neovascular form of the disease represents approximately 10 percent of the overall disease prevalence, but it is responsible for 90 percent of the severe vision loss. Neovascular age-related macular degeneration is characterized by choroidal neovascularization that invades the subretinal space, often leading to exudation and hemorrhage. If the condition is left untreated, damage to photoreceptors and loss of central vision usually result, and after several months to years, the vessels are largely replaced by a fibrovascular scar. Major risk factors include cigarette smoking, nutritional factors, cardiovascular diseases, and genetic markers, including genes regulating complement, lipid, angiogenic, and extracellular matrix pathways. While no medication is available for dry AMD, wet AMD is treated with VEGF inhibitors (ranibizumab, aflibercept or pegaptanib).

Idiopathic orbital inflammatory disease (IOD), also known as orbital pseudotumor, is a benign, non-granulomatous orbital inflammatory process characterized by extraocular orbital and adnexal inflammation. Corticosteroids are the main treatment of IOD. Other alternative or adjuvant treatment options are NSAIDs, cytotoxic drugs (chlorambucil, cyclophosphamide), corticosteroid-sparing immunosuppressants (methotrexate, cyclosporine, azathioprine) and TNF-α inhibitors.

Orbital (ocular) myositis is an idiopathic inflammatory disorder that usually manifests in the extraocular muscles, presents acutely, and is often unilateral. Signs and symptoms include pyroptosis, redness over the involved muscle, conjunctival chemosis, lid ptosis, eye pain that is worse with movement, and diplopia. Diagnosis is made according to clinical findings as well as extraocular muscle enlargement found on computed tomographic or MR images. The diagnosis of ocular (ocular) myositis falls within the overall classification of idiopathic orbital inflammatory diseases, defined as non-infective non-specific orbital inflammation without identifiable local or systemic causes. Orbital myositis typically responds to corticosteroids; however, nonsteroidal anti-inflammatory drugs may be helpful in mild cases (Costa et al., Curr Allergy Asthma Rep. 2009; 9(4):316-323).

Chorioretinal inflammation, also known as chorioretinitis, is an inflammation of the choroid and retina of the eye. It is a form of posterior uveitis. If only the choroid is inflamed, it is termed choroiditis. Chorioretinitis is usually treated with a combination of corticosteroids and antibiotics.

Keratitis is a condition in which the cornea becomes inflamed. There are several types of keratitis, classified according to the infective cause, environmental aetiology (exposure keratitis, photokeratitis), etc. The treatment depends on the cause of keratitis. Infectious keratitis is treated with antibacterials (levofloxacin, gatifloxacin, moxifloxacin or ofloxacin).

There is an unmet need for novel and effective methods of treatment of ocular inflammatory diseases, essentially devoid of side-effects.

### SUMMARY OF THE INVENTION

In some embodiments, this invention provides a method of treating, preventing or ameliorating blepharitis or symptoms thereof, comprising administering a topical pharmaceutical composition comprising from about 0.01% w/w to about 2% w/w tapinarof and a carrier suitable for topical administration.

In some embodiments, this invention provides a method of repelling, killing or inhibiting the growth of Demodex mites in or around the eye of a subject in need thereof comprising administering a topical pharmaceutical composition comprising from about 0.01% w/w to about 2% w/w tapinarof and a carrier suitable for topical administration.

In some embodiments, this invention provides a regimen for treating, preventing or ameliorating blepharitis comprising administering to the eye once daily or twice daily a therapeutically effective amount of a topical pharmaceutical composition comprising from about 0.01% w/w to about 2% w/w tapinarof and a carrier suitable for topical administration until remission or alleviation of the blepharitis.

In some embodiments, this invention provides a regimen for repelling, killing or inhibiting the growth of Demodex mites in or around the eye of a subject once daily or twice daily a therapeutically effective amount of a topical pharmaceutical composition comprising from about 0.01% w/w to about 2% w/w tapinarof and a carrier suitable for topical administration until the Demodex mites are repelled, killed, or inhibited.

In some embodiments, this invention provides a composition and a method of treatment of ocular inflammatory diseases or ocular degenerative disease by administration to a subject in need thereof an ophthalmic composition comprising from tapinarof and optionally at least one additional active agent and a carrier suitable for ophthalmic administration. In some embodiments, the ocular inflammatory disease is blepharitis.

In some embodiments, this invention provides a composition and a method of treatment of ocular inflammatory diseases or ocular degenerative disease by administration to a subject in need thereof a topical ophthalmic composition comprising from about 0.01% w/w to about 10.0% w/w tapinarof and a carrier suitable for ophthalmic administration. In some embodiments, the ocular inflammatory disease is blepharitis.

Also provided is a topical ophthalmic composition and a method of treatment of ocular inflammatory diseases or ocular degenerative disease, the composition further comprising from about 0.01% to about 10.0% w/w at least one additional active agent selected from a weak corticosteroid (loteprednol etabonate, prednisolone acetate, triamcinolone acetate), an immune suppressant (cyclosporin), an immune mediator (lifitegrast), an antimuscarinic agent (atropine, homatropine), a VGF inhibitor (ranibizumab, aflibercept, pegaptanib), an NSAID, a cytotoxic drug (chlorambucil, cyclophosphamide), a corticosteroid-sparing immunosuppressant (methotrexate, azathioprine), a TNF-α inhibitor, an antibiotic (levofloxacin, gatifloxacin, moxifloxacin, ofloxacin) and combinations thereof.

In some embodiments, the present invention also provides dosage forms and kits comprising the above compositions.

In some embodiments, the compositions, dosage forms, kits, implants and methods of this invention are suitable for the treatment, inhibition, prevention or alleviation of an ocular inflammatory disease or an ocular degenerative disease and exhibit synergistic and/or additive effects which allow reducing the amounts of the active agents in the compositions. In some embodiments, the ocular inflammatory disease is blepharitis.

### DETAILED DESCRIPTION OF THE INVENTION

This invention provides a composition and a method of treatment of ocular inflammatory diseases or ocular degeneration disease by administration to a subject in need thereof an ophthalmic composition comprising tapinarof and optionally at least one additional active agent and a carrier suitable for ophthalmic administration. In another embodiment, the composition is a topical composition. In another embodiment, the composition is a topical ophthalmic composition. In another embodiment, the composition is an intraocular injectable composition.

Tapinarof (3,5-dihydroxy-4-isopropyl-trans-stilbene) is a pharmaceutical active agent investigated for the treatment of atopic dermatitis, psoriasis and psoriatic disorders (Zang Y N, et al., Int J Clin Pharmacol Ther. 2016 February; 54(2):87-95). Tapinarof is a small molecule produced as a metabolite by the bacterium Photorhabdus luminescens, a constituent symbiote of the nematode Heterorhabditis, and has been shown to have antifungal and antibacterial properties. Tapinarof has been demonstrated to experimentally bind to aryl hydrocarbon receptors whose downstream effects include decreasing inflammatory cytokines, regulating skin barrier gene expression, and diminishing oxidative stress through multiple mechanisms. Studies have reported its safety, tolerability and efficacy in both atopic dermatitis and psoriasis.

In one embodiment, the present invention provides compositions, implants, dosage forms, kits and articles of manufacture that include tapinarof, optionally in combination with at least one additional active agent, for the treatment of an ocular inflammatory disease and/or ocular degenerative disease. The compositions can be administered to a subject in need thereof using a variety of ophthalmic compositions. The preferred route is the topical ophthalmic route and the preferred formulations are the drops, the cream, the ointment, the foam and the solution (see Examples 1-3).

In one embodiment, the present invention provides methods for the treatment, prevention, inhibition and/or amelioration of an ocular inflammatory disease and/or an ocular degeneration disease, comprising administering (a) a topical ophthalmic composition comprising from about 0.01% w/w to about 10% w/w tapinarof; or (b) an intraocular injectable composition comprising from about 3 mg/ml to about 80 mg/ml tapinarof.

In another embodiment, the topical composition (or topical ophthalmic composition) comprises from 0.01% w/w to 2% w/w tapinarof. In another embodiment, the composition comprises from 0.05% w/w to 2% w/w tapinarof. In another embodiment, the composition comprises from 0.05% w/w to 1.5% w/w tapinarof. In another embodiment, the composition comprises from 0.1% w/w to 1.5% w/w tapinarof. In another embodiment, the composition comprises from 0.1% w/w to 1.0% w/w tapinarof. In another embodiment, the composition comprises from 0.1% w/w to 2.0% w/w tapinarof. In another embodiment, the composition comprises from 0.5% w/w to 2% w/w tapinarof. In another embodiment, the composition comprises 0.01% w/w to 0.5% w/w tapinarof. In another embodiment, the composition comprises from 0.5% w/w to 1% w/w tapinarof. In another embodiment, the composition comprises from 1% w/w to 1.5% w/w tapinarof. In another embodiment, the composition comprises from 1.5% w/w to 2% w/w tapinarof. In another embodiment, the composition comprises from 2% w/w to 5% w/w tapinarof. In another embodiment, the composition comprises from 5% w/w to 10% w/w tapinarof. In another embodiment, the composition comprises from 0.1%, 0.2%, 0.3%, 0.4%, 0.5%, 0.6%, 0.7%, 0.8%, 0.9% or 1% w/w of tapinarof Each possibility represents a separate embodiment of the present invention.

In another embodiment, the injectable composition comprises from about 3 mg/ml to about 80 mg/ml tapinarof. In another embodiment, the intraocular injectable composition comprises from 3 mg/ml to 10 mg/ml tapinarof. In another embodiment, the intraocular injectable composition comprises from 10 mg/ml to 30 mg/ml tapinarof. In another embodiment, the intraocular injectable composition comprises from 20 mg/ml to 50 mg/ml tapinarof. In another embodiment, the intraocular injectable composition comprises from 30 mg/ml to 60 mg/ml tapinarof. In another embodiment, intraocular injectable composition comprises from 50 mg/ml to 80 mg/ml tapinarof. In another embodiment, the intraocular injectable composition comprises from 3 mg/ml to 20 mg/ml tapinarof. In another embodiment, the intraocular injectable composition comprises from 10 mg/ml to 20 mg/ml tapinarof Each possibility represents a separate embodiment of the present invention.

In some embodiments, the composition, implants, method, and/or kit of this invention comprise additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

A non-limiting example of weak corticosteroid includes loteprednol etabonate, prednisolone acetate, triamcinolone acetate or combination thereof. A non-limiting example of an immune suppressant includes cyclosporin. A non-limiting example of an immune mediator includes lifitegrast. A non-limiting example of an antimuscarinic agent includes atropine, homatropine or combination thereof. A non-limiting example of a VGF inhibitor includes ranibizumab, aflibercept, pegaptanib or combination thereof. A non-limiting example of an NSAID include nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone or a combination thereof. A non-limiting example of a TNF-α inhibitor includes adalimumab. A non-limiting example of a cytotoxic drug includes chlorambucil, cyclophosphamide or combination thereof. A non-limiting example of corticosteroid-sparing immunosuppressant include methotrexate, azathioprine or combination thereof. A non-limiting example of an antibiotic includes levofloxacin, gatifloxacin, moxifloxacin, ofloxacin, or combination thereof.

In one embodiment, the present invention also provides a topical combination composition comprising tapinarof and from about 0.01% w/w to about 10.0% w/w at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof and a carrier suitable for topical administration. Each possibility represents a separate embodiment of the present invention.

In another embodiment, the topical composition (which comprises also an additional active agent) comprises from 0.01% w/w to 2% w/w tapinarof. In another embodiment, the composition comprises from 0.05% w/w to 1.5% w/w tapinarof. In another embodiment, the composition comprises from 0.1% w/w to 1.5% w/w tapinarof. In another embodiment, the composition comprises from 0.1% w/w to 1.0% w/w tapinarof. In another embodiment, the composition comprises from 0.5% w/w to 2% w/w tapinarof. In another embodiment, the composition is a topical composition comprising from about 0.01% w/w to about 10.0% w/w tapinarof. In another embodiment, the topical composition comprises from 0.25% w/w to 0.5% tapinarof. In another embodiment, the topical composition comprises from 0.5% w/w to 1% tapinarof. In another embodiment, the topical composition comprises from 1% w/w to 1.5% tapinarof. In another embodiment, the topical composition comprises from 1.5% w/w to 2% tapinarof. In another embodiment, the topical composition comprises from 2% w/w to 5% tapinarof. In another embodiment, the topical composition comprises from 5% w/w to 10% tapinarof. In another embodiment, the composition comprises from 0.1%, 0.2%, 0.3%, 0.4%, 0.5%, 0.6%, 0.7%, 0.8%, 0.9% or 1% w/w of tapinarof. Each possibility represents a separate embodiment of the present invention.

In another embodiment, the composition (which comprises also an additional active agent) is an intraocular injectable composition comprising from about 3 mg/ml to 80 mg/ml tapinarof. In another embodiment, the intraocular injectable composition comprises from 3 mg/ml to 10 mg/ml tapinarof. In another embodiment, the intraocular injectable composition comprises from 10 mg/ml to 30 mg/ml tapinarof. In another embodiment, the intraocular injectable composition comprises from 20 mg/ml to 50 mg/ml tapinarof. In another embodiment, the intraocular injectable composition comprises from 30 mg/ml to 60 mg/ml tapinarof. In another embodiment, intraocular injectable composition comprises from 50 mg/ml to 80 mg/ml tapinarof. In another embodiment, the intraocular injectable composition comprises from 3 mg/ml to 20 mg/ml tapinarof. In another embodiment, the intraocular injectable composition comprises from 10 mg/ml to 20 mg/ml tapinarof. Each possibility represents a separate embodiment of the present invention.

In another embodiment, the intraocular injectable composition further comprises from 3 mg/ml to 80 mg/ml additional active agent. In another embodiment, the additional active agent is selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof and a carrier suitable for intraocular injectable administration. In another embodiment, the composition comprises from 3 mg/ml to 10 mg/ml additional active agent. In another embodiment, the composition comprises from 5 mg/ml to 15 mg/ml additional active agent. In another embodiment, the composition comprises from 10 mg/ml to 20 mg/ml additional active agent. In another embodiment, the composition comprises from 20 mg/ml to 30 mg/ml additional active agent. In another embodiment, the composition comprises from 30 mg/ml to 50 mg/ml additional active agent. In another embodiment, the composition comprises from 40 mg/ml to 80 mg/ml additional active agent. In another embodiment, the composition comprises from 10 mg/ml to 30 mg/ml additional active agent. Each possibility represents a separate embodiment of the present invention.

In some embodiments, tapinarof and optionally at least one additional active agent in the compositions of this invention are included in an amount effective for treating, preventing, inhibiting, or reducing the symptoms of an ocular inflammatory disease and/or ocular degeneration disease. The selection of the at least one additional active agent depends on the specific ocular inflammatory disease and/or ocular degeneration disease and the medical indication of the specific tapinarof/additional active agent combination. The concentrations of the active agents in the composition will depend on absorption, inactivation, excretion rates of the active compound, the synergistic or additive effects, the dosage schedule, and amount administered as well as other factors known to those of skill in the art. Generally, the dosages and concentrations will be lower, typically at least about or at 5 to 10% lower but up to about 15, 20, 25, 30, 35, 40, 50, 90 or 95% lower than the amount of tapinarof and optionally at least one additional active agent in the marketed single drug currently administered or considered for the treatment of an ocular inflammatory disease and/or an ocular degeneration disease. The dosage and regimen of administration may be determined by dose finding studies, as known in the art. Each possibility represents a separate embodiment of the present invention.

Exemplary dosages, strengths and concentrations of tapinarof in the tapinarof compositions administered topically, can be in the range of from about or at 0.1%, 0.25%, 0.5%, 1%, 2%, 3%, 4%, 5%, 6%, 7%, 8%, 9% or 10% w/w. Typical strengths in the topical combination compositions of this invention are 0.01%, 0.1%, 0.25%, 0.5%, 1% or 2% w/w tapinarof. The frequency of administration can be determined empirically. Exemplary frequencies are once daily, twice daily, three times per day, or four times per day, weekly, bi-weekly or monthly. Typical administration frequencies of the topical combination compositions of this invention are once daily and twice daily.

Exemplary dosages, strengths and concentrations of tapinarof in the intraocular injectable composition, can be in the range of from about or at 3 mg/ml, 5 mg/ml, 10 mg/ml, 15 mg/ml, 20 mg/ml, 25 mg/ml, 30 mg/ml, 40 mg/ml, 50 mg/ml, 60 mg/ml, 70 mg/ml or 80 mg/ml. Typical strengths in the combination compositions of this invention are 03 mg/ml, 5 mg/ml, 10 mg/ml, 15 mg/ml, 20 mg/ml, 25 mg/ml, 30 mg/ml, 40 mg/ml or 50 mg/ml tapinarof. The frequency of administration can be determined empirically. Exemplary frequencies are once daily, twice weekly, once weekly, twice monthly, monthly, every four to six weeks for a period of 4, 6, 8 or 12 months.

Dosage frequencies can be gradually attenuated over time and maintained at a steady dose suitable for long-term—six months, 1 year, 5 years, 10 years or more, up to lifelong administration to control the symptoms of the ocular inflammatory disease and/or of the ocular degeneration disease. For example, dosage administration can begin at from twice a day, to once a day, to two times a week, to once a week, to once every two weeks or less frequent than once every two weeks.

Pharmaceutical carriers or vehicles suitable for preparation of the topical ophthalmic compositions provided herein include any such carriers known to those skilled in the art to be suitable for the particular mode of administration. In some embodiments, a carrier comprises water, aqueous solvents, oily solvents, mixture of oily and aqueous solvents, thickeners, preservatives, bactericides, antioxidants or any combination thereof.

Pharmaceutical carriers or vehicles suitable for preparation of intraocular injectable compositions provided herein include any such carriers known to those skilled in the art to be suitable for the particular mode of administration. In some embodiments, a carrier comprises water, aqueous solvents, oily solvents, mixture of oily and aqueous solvents, thickeners, preservatives, bactericides, antioxidants or any combination thereof.

A non-limiting example of an antioxidant includes butylated hydroxytoluene, ascorbic acid, ascorbic palmitate, butylated hydroxyanisole, 2,4,5-trihydroxybutyrophenone, 4-hydroxymethyl-2,6-di-tert-butyl phenol, erythorbic acid, gum guaiac, propyl gallate, thiodipropionic acid, dilauryl thiodipropionate, tert-butylhydroquinone, tocopherol, sodium metabisulfite, sodium sulfite, or any combination thereof. In another embodiment, the antioxidant is present in a concentration of about 0.01% (w/w) to about 1% (w/w). In another embodiment, the antioxidant is present in a concentration of about 0.01% (w/w) to about 0.5% (w/w). In another embodiment, the antioxidant is present in a concentration of about 0.05% (w/w) to about 0.5% (w/w). In another embodiment, the antioxidant is present in a concentration of about 0.075% (w/w) to about 0.2% (w/w). In some embodiments, the composition comprises an antioxidant. In some embodiments, the composition does not include an antioxidant.

The resulting topical composition may be a lotion, a solution, a suspension, an emulsion or the like and is formulated as drops, creams, gels, ointments, emulsions, solutions, elixirs, lotions, suspensions, tinctures, pastes, foams, aerosols, transdermal patch sprays, foams, or any other formulation suitable for topical ophthalmic administration. The preferred topical compositions are the drops, the cream, the ointment, the solution, the foam and the lotion. In another embodiment, the topical composition includes nanomicelles, nanospheres or nanoparticles comprising an active agent (e.g. tapinarof).

In some embodiments, the topical composition is an aqueous ophthalmic solution, wherein the solution comprises nanomicelles.

In some embodiments, the topical composition is an ophthalmic solution, wherein the solution comprises nanomicelles, nanospheres or nanoparticles of an active agent (tapinarof).

In some embodiments, the topical composition is an ophthalmic solution, wherein the solution comprises nanomicelles, nanospheres or nanoparticles comprising tapinarof and optionally at least additional one active agent. In another embodiment, the tapinarof is loaded within the nanoparticles or nanospheres. In some embodiments, the active agent is encapsulated or non-encapsulated. In some embodiments, tapinarof is encapsulated or non-encapsulated.

Pharmaceutical carriers or vehicles suitable for administration of the active agents provided herein include any such carriers known to those skilled in the art to be suitable for the particular mode of administration. In addition, the active agent tapinarof may be formulated as the sole pharmaceutically active agent in the composition or may be combined with at least one additional active agent. The active agents are included in the carrier in an amount sufficient to exert a therapeutically useful effect i.e., amelioration of the symptoms of the ocular inflammatory disease and/or of the ocular degeneration disease, with minimal or no toxicity or other side effects. Generally, emollient or lubricating vehicles that help hydrate the eye are more preferred than volatile vehicles, such as ethanol, that dry the eye. Exemplary ingredients or vehicles for preparing compositions for use with topical ophthalmic compositions, (all within the regulatorily approved concentration ranges) are petrolatum, light mineral oil, lanolin, white wax, PEG 400, glycerin and cellulose derivatives. Suitable vehicles for intraocular injectable compositions of this invention are water or water and saline solutions.

Suitable pharmaceutically and dermatologically acceptable vehicles for topical application include those suited for use in drops, lotions, creams, ointments, solutions, gels, foams and the like. Generally, the vehicle is either organic in nature or an aqueous emulsion and capable of including the selected compound or compounds, which may be micronized, dispersed, suspended or dissolved therein. The vehicle may include pharmaceutically-acceptable emollients, moisturizers, penetration enhancers, fragrances and emulsifiers. In another embodiment, the topical composition includes nanomicelles, nanospheres or nanoparticles comprising an active agent (e.g. tapinarof). In another embodiment, the tapinarof and optionally the additional active agent are loaded within the nanoparticles or nanospheres, wherein the nanoparticles or nanospheres may be used as a drug carrier.

The compositions according to the invention are pharmaceutical compositions, and especially topical ophthalmic compositions, which may be in any form conventionally used for topical ophthalmic application. By addition of a fatty or oily phase, they may also be in the form of dispersions of the lotion or serum type, emulsions of liquid or semi-liquid consistency obtained by dispersing a fatty phase in an aqueous phase (O/W) or conversely (W/O), or suspensions or emulsions of soft, semi-liquid or solid consistency of the cream, gel or ointment type, or alternatively multiple emulsions (W/O/W or O/W/O), microemulsions, microparticles or vesicular dispersions of ionic and/or nonionic type, or wax/aqueous phase dispersions.

In some embodiments, said water in said oil in water emulsion further comprises at least one water soluble humectant.

In other embodiments, said at least one water soluble humectant is selected from the group consisting of propylene glycol, glycerin, and polyethylene glycol-X, where X is in the range of 200 to 10,000. Each possibility represents a separate embodiment of the present invention.

In further embodiments, there is provided a composition of the invention comprising, as a single pharmaceutical active agent, tapinarof for topical ophthalmic use or formulated for intraocular injection in the treatment of an ocular inflammatory disease and/or of an ocular degeneration disease. In some embodiments, the composition of the present invention comprises tapinarof and at least one additional active agent, wherein said additional active agent is selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

In further embodiments, the compositions of this invention for treating the ocular inflammatory disease and/or the ocular degeneration disease are controlled or slowed release drug delivery system, wherein the active agent is encapsulated, coated, adsorbed, embedded, impregnated, dispersed, entrapped, or encased in a polymeric material and providing a sustained release formulation. In another embodiment, the active agent(s) are encapsulated in a liposome. In another embodiment, the active agent is encapsulated in nanomicelles, or the active agent is loaded in nanoparticles or nanospheres. In another embodiment, the tapinarof and optionally the additional active agent are loaded within the nanoparticles or nanospheres, wherein the nanoparticles or nanospheres may be used as a drug carrier.

In other embodiments, microcapsules are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 2006/003014, US 2010/180464.

When referring to a “controlled or slowed release drug delivery system” it should be understood to relate to a delivery system (which in the present invention is a topical delivery system) that enables the release of the pharmaceutical active agent in predetermined amounts over a specified period.

### Methods of Treatment

According to an aspect of the invention, there is provided a method of treatment of an ocular inflammatory disease and/or an ocular degeneration disease, comprising administering to a subject in need thereof a therapeutically effective amount of an ophthalmic composition comprising and optionally at least one additional active agent. In another embodiment, the composition is a topical ophthalmic composition. In another embodiment, the composition is an intraocular injectable composition. According to an aspect of the invention, there is provided a method of treatment of an ocular inflammatory disease and/or an ocular degeneration disease, comprising administering to a subject in need thereof a therapeutically effective amount of an ophthalmic composition comprising tapinarof and optionally at least one additional active agent. In another embodiment, the composition is a topical ophthalmic composition. In another embodiment, the composition is an intraocular injectable composition.

In some embodiments, the therapeutically effective amount is an effective amount of tapinarof and optionally at least one additional active agent, namely an amount which will cure, treat, mitigate and/or prevent the ocular inflammatory disease and/or the ocular degeneration disease.

In some embodiments, provided herein is a method of treating, preventing or ameliorating blepharitis or symptoms thereof, comprising administering a topical pharmaceutical composition comprising from about 0.01% w/w to about 2% w/w at least one acaricidal active agent and a carrier suitable for topical administration. In other embodiments, the—acaricidal active agent is selected from the group consisting of ivermectin, permethrin, niclosamide, benzoyl peroxide, tapinarof and combinations thereof; wherein at least one of the acaricidal active agent is tapinarof. In other embodiments, the at least one acaricidal active agent is tapinarof. In other embodiments, the blepharitis symptoms are selected from redness, irritation, burning, itching, tearing, crusting of eyelashes, and madarosis.

In some embodiments, provided herein is a method of repelling, killing or inhibiting the growth of Demodex mites in or around the eye of a subject in need thereof comprising administering a topical pharmaceutical composition comprising from about 0.01% w/w to about 2% w/w at least one acaricidal active agent and a carrier suitable for topical administration. In other embodiments, the acaricidal active agent is selected from the group consisting of ivermectin, permethrin, niclosamide, benzoyl peroxide, tapinarof and combinations thereof; wherein at least one of the acaricidal active agent is tapinarof. In other embodiments, the at least one acaricidal active agent is tapinarof.

In some embodiment, repelling, killing, inhibiting the growth or removing Demodex mites refers herein to treating Demodex mites.

Acaricides are pesticides that kill members of the arachnid subclass of Acari parasites. Acari includes mites and ticks. Mites and ticks are usually treated as a single group. A less common name is “miticides” which are substances killing mites.

The purpose of the at least one acaricide component of the composition is to kill the mites of the Demodex folliculorum, which is one of the causes of rosacea. A number of acaricides are commercially available, the most prominent being permethrin, ivermectin and crotamiton. For the purpose of this invention, acaricides (miticides) potentially active against Demodex mites are selected. The at least one acaricidal active agent in the composition of this invention is selected from ivermectin, permethrin, niclosamide, benzoyl peroxide, tapinarof and combinations thereof.

In some embodiments, provided herein a method of treating, preventing or ameliorating blepharitis or symptoms thereof, comprising administering a topical pharmaceutical composition comprising from about 0.01% w/w to about 2% w/w tapinarof and a carrier suitable for topical administration. In other embodiments, the blepharitis symptoms are selected from redness, irritation, burning, itching, tearing, crusting of eyelashes, and madarosis.

In some embodiments, the blepharitis is selected from anterior inflammatory blepharitis or posterior inflammatory blepharitis. In other embodiments, the blepharitis treatment described herein is involved with Demodex mites. In other embodiments, the method disclosed herein for treating blepharitis is further repels, kills or inhibits the growth of Demodex mites in or around the eye. In other embodiments, the Demodex mites comprise Demodex brevis or Demodex folliculorum.

In some embodiments, provided herein is a method of repelling, killing or inhibiting the growth of demodex mites in or around the eye of a subject in need thereof comprising administering a topical pharmaceutical composition comprising from about 0.01% w/w to about 2% w/w tapinarof and a carrier suitable for topical administration. In other embodiments, Demodex mites disclosed herein comprise Demodex brevis or Demodex folliculorum.

In one embodiment, the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital (ocular) myositis, and combinations thereof. In another embodiment the ocular inflammatory disease and/or the ocular degeneration disease is uveitis. In another embodiment the ocular inflammatory disease and/or the ocular degeneration disease is vitritis. In another embodiment the ocular inflammatory disease and/or the ocular degeneration disease is dry eye disease (DED). In another embodiment the ocular inflammatory disease and/or the ocular degeneration disease is macular degeneration. In another embodiment the ocular inflammatory disease and/or the ocular degeneration disease is idiopathic orbital inflammatory disease (IOD). In another embodiment the ocular inflammatory disease and/or the ocular degeneration disease is blepharitis. In another embodiment the ocular inflammatory disease and/or the ocular degeneration disease is seborrheic dermatitis of eyelids. In another embodiment the ocular inflammatory disease and/or the ocular degeneration disease is seborrheic dermatitis of eyebrows. In another embodiment the ocular inflammatory disease and/or the ocular degeneration disease is hyperemia. In another embodiment the ocular inflammatory disease and/or the ocular degeneration disease is thyroid eye disease (TED), In another embodiment the ocular inflammatory disease and/or the ocular degeneration disease is chorioretinal inflammation. In another embodiment the ocular inflammatory disease and/or the ocular degeneration disease is keratitis. In another embodiment the ocular inflammatory disease and/or the ocular degeneration disease is age-related macular degeneration (AMD). In another embodiment, the ocular inflammatory disease and/or the ocular degeneration disease is orbital (ocular) myositis.

In some embodiments, co-administration of tapinarof and at least one additional active agent in one composition or two separate compositions administered sequentially or simultaneously, in either order, exhibits an additive and/or synergistic effect while treating, preventing or alleviating ocular inflammatory disease and/or ocular degeneration disease.

In some other embodiments, the co-administration may be made either by administration of a single combination composition, or alternatively by separate administration of a first composition comprising tapinarof and a second composition comprising at least one additional active agent.

Regimen of Administration of Ophthalmic Tapinarof Compositions for Treatment of an Ocular Inflammatory Disease and/or an Ocular Degenerative Disease

Therapeutically effective concentrations of tapinarof and optionally at least one additional active agent in the compositions of this invention for treatment, prevention, inhibition, or amelioration of an ocular inflammatory disease or an ocular degenerative disease or of the symptoms manifested by the ocular inflammatory disease and/or the ocular degenerative disease are determined by empirical methods known in the art.

In some embodiments, provided herein a regimen for treating, preventing or ameliorating blepharitis comprising administering to the eye once daily or twice daily a therapeutically effective amount of a topical pharmaceutical composition comprising from about 0.01% w/w to about 2% w/w at least one acaricidal active agent and a carrier suitable for topical administration until remission or alleviation of the blepharitis. In other embodiments, the—acaricidal active agent is selected from the group consisting of ivermectin, permethrin, niclosamide, benzoyl peroxide, tapinarof and combinations thereof; wherein at least one of the acaricidal active agent is tapinarof. In other embodiments, the at least one acaricidal active agent is tapinarof.

In some embodiments, this invention provides a regimen for treating, preventing or ameliorating blepharitis comprising administering to the eye once daily or twice daily a therapeutically effective amount of a topical pharmaceutical composition comprising from about 0.01% w/w to about 2% w/w tapinarof and a carrier suitable for topical administration until remission or alleviation of the blepharitis. In other embodiments, the regimen comprises topically applying the pharmaceutical composition to the cutaneous tissue surrounding the eyelash or eyelid and/or to the eyelash or the eyelid, or to the eyelid margin.

In some embodiments, provided herein a regimen for repelling, killing or inhibiting the growth of Demodex mites in or around the eye of a subject once daily or twice daily a therapeutically effective amount of a topical pharmaceutical composition comprising from about 0.01% w/w to about 2% w/w at least one acaricidal active agent and a carrier suitable for topical administration until the Demodex mites are repelled, killed, or inhibited. In other embodiments, the—acaricidal active agent is selected from the group consisting of ivermectin, permethrin, niclosamide, benzoyl peroxide, tapinarof and combinations thereof; wherein at least one of the acaricidal active agent is tapinarof. In other embodiments, the at least one acaricidal active agent is tapinarof.

In some embodiments, provided herein a regimen for repelling, killing or inhibiting the growth of Demodex mites in or around the eye of a subject once daily or twice daily a therapeutically effective amount of a topical pharmaceutical composition comprising tapinarof and a carrier suitable for topical administration until the Demodex mites are repelled, killed, or inhibited. In other embodiments, the regimen comprises topically applying the pharmaceutical composition to the cutaneous tissue surrounding the eyelash or eyelid and/or to the eyelash or the eyelid, or to the eyelid margin.

The concentration of the additional active agents in the composition will depend on absorption, inactivation, excretion rates of the active compound, synergistic and/or additive effects, the dosage schedule, and amount administered as well as other factors known to those of skill in the art. Generally, the dosages and concentrations will be lower, typically at least about or at 5 to 10% lower but up to about or at 15, 20, 25, 30, 35, 40, 50, 90 or 95% lower than the amount of tapinarof and at least one additional active agent in the developed or marketed single drug currently being developed or used for the treatment of the ocular inflammatory disease and/or the ocular degeneration disease. The dosage and regimen of administration may be determined by dose finding studies, as known in the art.

Exemplary dosages, strengths and concentrations of tapinarof in the tapinarof compositions administered topically, can be in the range of from about 0.01%, 0.1%, 0.25%, 0.5%, 1%, 2%, 3%, 4%, 5%, 6%, 7%, 8%, 9% or 10% w/w.

Typical tapinarof strengths in the topical combination compositions of this invention are 0.01%, 0.1%, 0.25%, 0.5%, 1%, 2%, 5% or 10% w/w tapinarof.

The frequency of administration can be determined empirically. Exemplary frequencies are once daily, twice daily, weekly, bi-weekly or monthly.

Typical administration frequencies of the topical combination compositions of this invention are once daily and twice daily.

Exemplary dosages, strengths and concentrations of tapinarof in the intraocular injectable composition, can be in the range of from about or at 3 mg/ml, 5 mg/ml, 10 mg/ml, 15 mg/ml, 20 mg/ml, 25 mg/ml, 30 mg/ml, 40 mg/ml, 50 mg/ml, 60 mg/ml, 70 mg/ml or 80 mg/ml. Typical strengths in the combination compositions of this invention are 03 mg/ml, 5 mg/ml, 10 mg/ml, 15 mg/ml, 20 mg/ml, 25 mg/ml, 30 mg/ml, 40 mg/ml or 50 mg/ml tapinarof. The frequency of administration can be determined empirically. Exemplary frequencies are once daily, twice weekly, once weekly, twice monthly, monthly, every four to six weeks for a period of 4, 6, 8 or 12 months.

Dosage frequencies can be gradually attenuated over time and maintained at a steady dose suitable for long-term—six months, 1 year, 5 years, 10 years or more, up to lifelong administration to control the symptoms of an ocular inflammatory disease and/or of an ocular degeneration disease. For example, dosage administration can begin at from twice a day, to once a day, to two times a week, to once a week, to once every two weeks or less frequent than once every two weeks.

### Kits

There are provided kits containing the dosage forms and compositions of this invention, optionally including instructions for administration.

The compositions provided herein can be packaged as articles of manufacture containing packaging material, a composition provided herein, and a label that indicates that the composition is for treating an ocular inflammatory disease and/or an ocular degenerative disease and/or for treating Demodex mites, and is formulated for topical delivery or topical ophthalmic. In another embodiment, the ocular inflammatory disease is blepharitis.

The articles of manufacture provided herein contain packaging materials. Packaging materials for use in packaging pharmaceutical products are well known to those of skill in the art. Examples of pharmaceutical packaging materials include, but are not limited to drop dispensers, bottles, tubes, containers, application syringes and any packaging material suitable for the selected formulation and intended mode of administration and treatment.

### Implant

In some embodiments this invention provides an implant comprising tapinarof for use in treating ocular inflammatory disease and/or an ocular degeneration disease.

In some embodiments this invention provides an implant comprising tapinarof for use in treating age-related macular degeneration.

In another embodiment, the implant is implanted in the retina of the eye. In another embodiment, the implant comprises a layer of a composition comprising tapinarof and optionally with at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

In some embodiments this invention provides an implant comprising from about 0.5 mg to about 80 mg tapinarof. In some embodiments this invention provides an implant comprising from about 0.5 mg to about 80 mg tapinarof and optionally from about 0.5 mg to 10 mg additional active agent.

In another embodiment, the implant is a plastic rod with a pellet comprising tapinarof (or optionally with at least one additional active agent) on the end. The pellet slowly dissolves and releases the medication into the fluid in the eye. In another embodiment, the implant can release the medicine (Tapinarof) for 2 to 4 years.

### EMBODIMENTS

In some embodiments, there is provided an ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration or from about 3 mg/ml to about 80 mg/ml tapinarof and a carrier suitable for intraocular injectable administration. In another embodiment, the ocular inflammatory disease is blepharitis.

In some embodiments, there is provided a topical ophthalmic composition for the treatment, prevention and/or amelioration of an ocular inflammatory disease and/or an ocular degenerative disease, comprising from about 0.01% w/w to about 10.0% w/w tapinarof and a carrier suitable for topical ophthalmic administration. In another embodiment, the ocular inflammatory disease is blepharitis.

In some embodiments, there is provided an intraocular injectable composition for the treatment, prevention and/or amelioration of an ocular inflammatory disease and/or an ocular degenerative disease, comprising from about 3 mg/ml to about 80 mg/ml tapinarof and a carrier suitable for intraocular injectable administration. In another embodiment, the ocular inflammatory disease is blepharitis.

In some other embodiments, there is provided a topical ophthalmic composition for the treatment, prevention and/or amelioration of an ocular inflammatory disease and/or an ocular degenerative disease, comprising from about 0.01% w/w to about 10.0% w/w tapinarof and a carrier suitable for topical ophthalmic administration, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In some other embodiments, there is provided an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injectable administration, wherein the ocular inflammatory disease and/or the ocular degeneration disease is selected from uveitis, vitritis, dry eye disease (DED), macular degeneration, idiopathic orbital inflammatory disease (IOD), blepharitis, seborrheic dermatitis of eyelids, seborrheic dermatitis of eyebrows, hyperemia, thyroid eye disease (TED), chorioretinal inflammation, keratitis, age-related macular degeneration (AMD), orbital myositis and combinations thereof. In another embodiment, the ocular inflammatory disease is blepharitis.

In other embodiments, blepharitis is anterior inflammatory blepharitis. In other embodiments, blepharitis is posterior inflammatory blepharitis. In some other embodiments, blepharitis is with involvement of Demodex mites. In some other embodiments, blepharitis is without involvement of Demodex mites.

According to some embodiments, there is provided a topical ophthalmic composition for the treatment, prevention and/or amelioration of an ocular inflammatory disease and/or an ocular degenerative disease, comprising from about 0.01% w/w to about 10.0% w/w tapinarof and a carrier suitable for topical ophthalmic administration, further comprising from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

According to some embodiments, there is provided an intraocular injectable composition for the treatment, prevention and/or amelioration of an ocular inflammatory disease and/or an ocular degenerative disease, comprising from about 3 mg/ml to about 80 mg/ml tapinarof and a carrier suitable for intraocular injectable administration, further comprising from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

According to some embodiments, there is provided an intraocular injectable composition for the treatment, prevention and/or amelioration of an ocular inflammatory disease and/or an ocular degenerative disease, comprising from about 3 mg/ml to about 80 mg/ml tapinarof and a carrier suitable for intraocular injectable administration, further comprising from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof; wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis and combinations thereof. In another embodiment, the ocular inflammatory disease is blepharitis.

In other embodiments, blepharitis is anterior inflammatory blepharitis. In other embodiments, blepharitis is posterior inflammatory blepharitis. In some other embodiments, blepharitis is with involvement of Demodex mites. In some other embodiments, blepharitis is without involvement of Demodex mites.

According to some embodiments, there is provided an intraocular injectable composition for the treatment, prevention and/or amelioration of age-related macular degeneration (AMD), comprising from about 3 mg/ml to about 80 mg/ml tapinarof and a carrier suitable for intraocular injectable administration. In another embodiment, the age-related macular degeneration is neovascular age-related macular degeneration (wet). In another embodiment, the age-related macular degeneration is geographic atrophy (late dry).

According to some embodiments, there is provided an intraocular injectable composition for the treatment, prevention and/or amelioration of age-related macular degeneration (AMD), comprising from about 3 mg/ml to about 80 mg/ml tapinarof and further comprising from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

According to some other embodiments, there is provided a topical ophthalmic composition for the treatment, prevention and/or amelioration of an ocular inflammatory disease and/or an ocular degenerative disease, comprising from about 0.01% w/w to about 10.0% w/w tapinarof and a carrier suitable for topical ophthalmic administration, further comprising from about 0.01% w/to about 10% w/w at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

According to some other embodiments, there is provided an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injectable administration, further comprising from about 3 mg/ml to about 80 mg/ml at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

In some embodiments, there is provided a dosage form for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, wherein the dosage form is selected from an ointment, a suspension, a cream, a spray, a lotion, a gel, an emulsion, a solution (including nanomicellar solutions), an elixir, a tincture, a paste, a foam and drops. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a dosage form for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof, and wherein the dosage form is selected from an ointment, a suspension, a cream, a spray, a lotion, a gel, an emulsion, a solution (including nanomicellar solutions), an elixir, a tincture, a paste, a foam and drops. In another embodiment the tapinarof is formulated within nanomicelles, nanospheres or nanoparticles. In another embodiment, the tapinarof is loaded within the nanomicelles, nanoparticles or nanospheres.

In other embodiments, blepharitis is anterior inflammatory blepharitis. In other embodiments, blepharitis is posterior inflammatory blepharitis. In some other embodiments, blepharitis is with involvement of Demodex mites. In some other embodiments, blepharitis is without involvement of Demodex mites.

In some embodiments, there is provided a dosage form for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, and optionally from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the dosage form is selected from an ointment, a suspension, a cream, a spray, a lotion, a gel, an emulsion, a solution, an elixir, a tincture, a paste, a foam and drops. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a dosage form for the treatment, prevention and/or amelioration of an ocular inflammatory disease and/or an ocular degeneration disease, comprising from about 0.01% w/w to about 10.0% w/w tapinarof and a carrier suitable for topical ophthalmic administration, and optionally from about 0.01% w/to about 10% w/w at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof, wherein the dosage form is selected from an ointment, a suspension, a cream, a spray, a lotion, a gel, an emulsion, a solution (including nanomicellar solutions), an elixir, a tincture, a paste, a foam and drops. In another embodiment the tapinarof is formulated within nanomicelles, nanospheres or nanoparticles. In another embodiment, the tapinarof is loaded within the nanomicelles, nanoparticles or nanospheres.

In some embodiments, provided herein a dosage form for treating, preventing or ameliorating blepharitis or symptoms thereof comprising a topical pharmaceutical composition comprising from about 0.01% w/w to about 2% w/w at least one acaricidal active agent and a carrier suitable for topical administration wherein the composition is formulated in a dosage form selected from an ointment, suspension, solution, drops, cream, lotion, gel, aerosol, transdermal patch, spray, or foam. In other embodiments, the acaricidal active agent is selected from the group consisting of ivermectin, permethrin, niclosamide, benzoyl peroxide, tapinarof and combinations thereof; wherein at least one of the acaricidal active agent is tapinarof. In another embodiment, the at least one acaricidal active agent is tapinarof.

In some embodiments, provided herein a dosage form for treating, preventing or ameliorating blepharitis or symptoms thereof comprising a topical pharmaceutical composition comprising from about 0.01% w/w to about 2% w/w tapinarof and a carrier suitable for topical administration wherein the composition is formulated in a dosage form selected from an ointment, suspension, solution, drops, cream, lotion, gel, aerosol, transdermal patch, spray, or foam.

In some embodiments, provided herein a dosage form for repelling, killing or inhibiting the growth of Demodex mites in or around the eye of a subject comprising a topical pharmaceutical composition comprising from about 0.01% w/w to about 2% w/w at least one acaricidal active agent and a carrier suitable for topical administration wherein the composition is formulated in a dosage form selected from an ointment, suspension, solution, drops, cream, lotion, gel, aerosol, transdermal patch, spray, or foam. In other embodiments, the acaricidal active agent is selected from the group consisting of ivermectin, permethrin, niclosamide, benzoyl peroxide, tapinarof and combinations thereof; wherein at least one of the acaricidal active agent is tapinarof. In another embodiment, the at least one acaricidal active agent is tapinarof.

In some embodiments, provided herein a dosage form for repelling, killing or inhibiting the growth of Demodex mites in or around the eye of a subject comprising a topical pharmaceutical composition comprising from about 0.01% w/w to about 2% w/w tapinarof and a carrier suitable for topical administration wherein the composition is formulated in a dosage form selected from an ointment, suspension, solution, drops, cream, lotion, gel, aerosol, transdermal patch, spray, or foam.

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration, wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a method of treatment, prevention and/or alleviation of an ocular inflammatory disease and/or an ocular degeneration disease, comprising administering to the eyes of a subject in need thereof a therapeutically effective amount of an intraocular injectable composition comprising from about 3 mg/ml to about 80 mg/ml tapinarof and a carrier suitable for intraocular injectable administration.

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof, and wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a method of treatment, prevention and/or alleviation of an ocular inflammatory disease and/or an ocular degeneration disease comprising administering to the eyes of a subject in need thereof a therapeutically effective amount of an intraocular injectable composition comprising from about 3 mg/ml to about 80 mg/ml tapinarof and a carrier suitable for intraocular injectable administration, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In another embodiment, the ocular inflammatory disease is blepharitis.

In some embodiments, there is provided a method of treatment, prevention and/or alleviation of age-related macular degeneration (AMD), comprising administering to the eyes of a subject in need thereof a therapeutically effective amount of an intraocular injectable composition comprising from about 3 mg/ml to about 80 mg/ml tapinarof and a carrier suitable for intraocular injectable administration. In another embodiment, the age-related macular degeneration is neovascular age-related macular degeneration (wet). In another embodiment, the age-related macular degeneration is geographic atrophy (late dry).

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration, and optionally from about 0.01% w/to about 10% w/w at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof and wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof, and wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres. In another embodiment, the ocular inflammatory disease is blepharitis.

In other embodiments, blepharitis is anterior inflammatory blepharitis. In other embodiments, blepharitis is posterior inflammatory blepharitis. In some other embodiments, blepharitis is with involvement of Demodex mites. In some other embodiments, blepharitis is without involvement of Demodex mites.

In some embodiments, there is provided a method of treatment, prevention and/or alleviation of an ocular inflammatory disease and/or an ocular degeneration disease, comprising administering to the eyes of a subject in need thereof a therapeutically effective amount of an intraocular injectable composition comprising from about 3 mg/ml to about 80 mg/ml tapinarof and a carrier suitable for intraocular injection, and optionally from about 3 mg/ml to about 80 mg/ml at least one additional active agent selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof and wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In another embodiment, the ocular inflammatory disease is blepharitis.

In other embodiments, blepharitis is anterior inflammatory blepharitis. In other embodiments, blepharitis is posterior inflammatory blepharitis. In some other embodiments, blepharitis is with involvement of Demodex mites. In some other embodiments, blepharitis is without involvement of Demodex mites.

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration and optionally from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres.

In some embodiments, there is provided a method of treatment, prevention and/or alleviation of an ocular inflammatory disease and/or an ocular degeneration disease, comprising administering to the eyes of a subject in need thereof a therapeutically effective amount of intraocular injectable composition comprising from about 3 mg/ml to about 80 mg/ml tapinarof and a carrier suitable for intraocular injectable administration, and optionally from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof.

In some embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration and optionally from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), orbital myositis, and combinations thereof, and wherein the composition is formulated as an ophthalmic ointment, suspension, a spray, a lotion, a gel, emulsion, solution (including nanomicellar solutions), an elixir, a tincture, a paste, drops, cream or foam. In another embodiment the tapinarof is formulated within nanomicelles, nanospheres or nanoparticles. In another embodiment, the tapinarof is loaded within the nanomicelles, nanoparticles or nanospheres. In another embodiment, the ocular inflammatory disease is blepharitis.

In some embodiments, there is provided a method of treatment, prevention and/or alleviation of an ocular inflammatory disease and/or an ocular degeneration disease, comprising administering to the eyes of a subject in need thereof a therapeutically effective amount of an intraocular injectable composition comprising from about 3 mg/ml to about 80 mg/ml tapinarof and a carrier suitable for intraocular injectable administration, and optionally from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, and wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In another embodiment, the ocular inflammatory disease is blepharitis.

In some other embodiments, there is provided a method of treatment, prevention and/or alleviation of an ocular inflammatory disease and/or an ocular degeneration disease, comprising topically administering to the eyes of a subject in need thereof a therapeutically effective amount of a composition comprising from about 0.01% w/w to about 10.0% w/w tapinarof and a carrier suitable for topical ophthalmic administration and optionally from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In another embodiment, the ocular inflammatory disease is blepharitis.

According to some embodiments, there is provided a method of treatment, prevention and/or alleviation of an ocular inflammatory disease and/or an ocular degeneration disease, wherein the treatment comprises once daily or twice daily topical administration to a subject in need thereof of a therapeutically effective amount of a composition comprising from about 0.01% w/w to about 10.0% w/w tapinarof and a carrier suitable for topical ophthalmic administration and optionally from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In another embodiment, the ocular inflammatory disease is blepharitis.

According to some embodiments, there is provided a method of treatment, prevention and/or alleviation of an ocular inflammatory disease and/or an ocular degeneration disease, wherein the treatment comprises once daily or twice daily topical administration to a subject in need thereof of a therapeutically effective amount of a composition comprising from about 0.01% w/w to about 10.0% w/w tapinarof and a carrier suitable for topical ophthalmic administration. In another embodiment, the ocular inflammatory disease is selected and/or the ocular degeneration disease from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In another embodiment, the ocular inflammatory disease is blepharitis.

According to some embodiments, there is provided a method of treatment, prevention and/or alleviation of an ocular inflammatory disease and/or an ocular degeneration disease, wherein the treatment comprises administering an intraocular injectable composition to a subject in need thereof comprising from about 3 mg/ml to about 80 mg/ml tapinarof and a carrier suitable for intraocular injectable composition. In another embodiment, the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In another embodiment, the ocular inflammatory disease is blepharitis.

According to some other embodiments, there is provided a method of treatment, prevention and/or alleviation of an ocular inflammatory disease and/or an ocular degeneration disease, wherein the treatment comprises once daily or twice daily topical administration to a subject in need thereof of a therapeutically effective amount of a composition comprising from about 0.01% w/w to about 10.0% w/w tapinarof and a carrier suitable for topical ophthalmic administration and from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof. In another embodiment, the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In another embodiment, the ocular inflammatory disease is blepharitis.

According to some embodiments, there is provided a method of treatment, prevention and/or alleviation of an ocular inflammatory disease and/or an ocular degeneration disease, wherein the treatment comprises administering an intraocular injectable composition to a subject in need thereof comprising from about 3 mg/ml to about 80 mg/ml tapinarof and a carrier suitable for intraocular injectable administration and from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof. In another embodiment, the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), age-related macular degeneration (AMD), orbital myositis, and combinations thereof. In another embodiment, the ocular inflammatory disease is blepharitis.

In other embodiments, blepharitis is anterior inflammatory blepharitis. In other embodiments, blepharitis is posterior inflammatory blepharitis. In some other embodiments, blepharitis is with involvement of Demodex mites. In some other embodiments, blepharitis is without involvement of Demodex mites.

In some embodiments, there is provided a method of treatment, prevention and/or alleviation of an ocular inflammatory disease and/or an ocular degeneration disease, wherein the treatment comprises once daily or twice daily topical administration to a subject in need thereof of a therapeutically effective amount of a composition comprising from about 0.01% w/w to about 10.0% w/w tapinarof and a carrier suitable for topical ophthalmic administration and optionally from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein tapinarof and the at least one additional active agent exhibit an additive or synergistic effect, thereby allowing to reduce the amounts of the active agents in the composition.

In some embodiments, there is provided a method of treatment, prevention and/or alleviation of an ocular inflammatory disease and/or an ocular degeneration disease, wherein the treatment comprises to a subject in need thereof a therapeutically effective amount of an intraocular injectable composition comprising from about 3 mg/ml to about 80 mg/ml tapinarof and a carrier suitable for intraocular injectable administration and optionally from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein tapinarof and the at least one additional active agent exhibit an additive or synergistic effect, thereby allowing to reduce the amounts of the active agents in the composition.

In some embodiments, there is provided a method of treating, preventing or ameliorating blepharitis or symptoms thereof, comprising administering a topical pharmaceutical composition comprising from about 0.01% w/w to about 2% w/w at least one acaricidal active agent and a carrier suitable for topical administration. In other embodiments, the at least one acaricidal active agent is selected from the group consisting of ivermectin, permethrin, niclosamide, benzoyl peroxide, tapinarof and combinations thereof; wherein at least one of the acaricidal active agent is tapinarof. In other embodiments, the at least one acaricidal active agent is tapinarof. In other embodiments, the method comprises topically applying the pharmaceutical composition to the cutaneous tissue surrounding the eyelash or eyelid and/or to the eyelash or the eyelid, or to the eyelid margin. In other embodiments the blepharitis is selected from anterior inflammatory blepharitis or posterior inflammatory blepharitis. In other embodiments, the composition is formulated as an ointment, suspension, spray, solution, drops, cream, lotion, gel, aerosol, transdermal patch, or foam. In other embodiments, the composition is applied by an applicator. In other embodiments, the blepharitis symptoms are selected from redness, irritation, burning, itching, tearing, crusting of eyelashes, and madarosis. In other embodiments, the composition comprises from about 0.05% w/w to about 1.5% w/w tapinarof and a carrier suitable for topical administration. In other embodiments, the composition comprises from about 0.1%, 0.5% or 1% w/w tapinarof and a carrier suitable for topical administration. In other embodiments, the tapinarof is encapsulated or non-encapsulated.

In some embodiments, there is provided a method of treating, preventing or ameliorating blepharitis or symptoms thereof, wherein the blepharitis is with involvement of demodex mites comprising administering a topical pharmaceutical composition comprising from about 0.01% w/w to about 2% w/w at least one acaricidal active agent and a carrier suitable for topical administration. In other embodiments, the at least one acaricidal active agent is selected from the group consisting of ivermectin, permethrin, niclosamide, benzoyl peroxide, tapinarof and combinations thereof; wherein at least one of the acaricidal active agent is tapinarof. In other embodiments, the Demodex mites comprise Demodex brevis or Demodex folliculorum. In other embodiments, the method comprises topically applying the pharmaceutical composition to the cutaneous tissue surrounding the eyelash or eyelid and/or to the eyelash or the eyelid, or to the eyelid margin. In other embodiments, the composition is formulated as an ointment, suspension, solution, cream, lotion, gel, spray. aerosol, transdermal patch, drops, spray or foam. In other embodiments, the composition is applied by an applicator. In other embodiments, the composition comprises from about 0.05% w/w to about 1.5% w/w tapinarof and a carrier suitable for topical administration. In other embodiments, the composition comprises from about 0.1%, 0.5% or 1% w/w tapinarof and a carrier suitable for topical administration. In other embodiments, the tapinarof is encapsulated or non-encapsulated.

In some embodiments, there is provided a method of treating, preventing or ameliorating blepharitis or symptoms thereof, comprising administering a topical pharmaceutical composition comprising from about 0.01% w/w to about 2% w/w tapinarof and a carrier suitable for topical administration. In other embodiments, the method comprises topically applying the pharmaceutical composition to the cutaneous tissue surrounding the eyelash or eyelid and/or to the eyelash or the eyelid, or to the eyelid margin. In other embodiments the blepharitis is selected from anterior inflammatory blepharitis or posterior inflammatory blepharitis. In other embodiments, the composition is formulated as an ointment, suspension, solution, drops, cream, lotion, gel, aerosol, transdermal patch, spray or foam. In other embodiments, the composition is applied by an applicator. In other embodiments, the blepharitis symptoms are selected from redness, irritation, burning, itching, tearing, crusting of eyelashes, and madarosis. In other embodiments, the composition comprises from about 0.05% w/w to about 1.5% w/w tapinarof and a carrier suitable for topical administration. In other embodiments, the composition comprises from about 0.1%, 0.5% or 1% w/w tapinarof and a carrier suitable for topical administration.

In some embodiments, there is provided a method of treating, preventing or ameliorating blepharitis or symptoms thereof, wherein the blepharitis is with involvement of Demodex mites comprising administering a topical pharmaceutical composition comprising from about 0.01% w/w to about 2% w/w tapinarof and a carrier suitable for topical administration. In other embodiments, the Demodex mites comprise Demodex brevis or Demodex folliculorum. In other embodiments, the method comprises topically applying the pharmaceutical composition to the cutaneous tissue surrounding the eyelash or eyelid and/or to the eyelash or the eyelid, or to the eyelid margin. In other embodiments, the composition is formulated as an ointment, suspension, solution, drops, cream, lotion, gel, aerosol, transdermal patch, spray or foam. In other embodiments, the composition is applied by an applicator. In other embodiments, the composition comprises from about 0.05% w/w to about 1.5% w/w tapinarof and a carrier suitable for topical administration. In other embodiments, the composition comprises from about 0.1%, 0.5% or 1% w/w tapinarof and a carrier suitable for topical administration. In other embodiments, the tapinarof is encapsulated or non-encapsulated.

In some embodiments, provided herein a method of repelling, killing or inhibiting the growth of Demodex mites in or around the eye of a subject in need thereof comprising administering a topical pharmaceutical composition comprising from about 0.01% w/w to about 2% w/w at least one acaricidal active agent and a carrier suitable for topical administration. In other embodiments, the at least one acaricidal active agent is selected from the group consisting of ivermectin, permethrin, niclosamide, benzoyl peroxide, tapinarof and combinations thereof; wherein at least one of the acaricidal active agent is tapinarof. In other embodiments, the at least one acaricidal active agent is tapinarof. In other embodiments, the method comprises topically applying the pharmaceutical composition to the cutaneous tissue surrounding the eyelash or eyelid and/or to the eyelash or the eyelid, or to the eyelid margin. In other embodiments, the Demodex mites comprise Demodex brevis or Demodex folliculorum. On other embodiments, the composition is formulated as an ointment, suspension, solution, drops, cream, lotion, gel, aerosol, transdermal patch, spray or foam. In other embodiments the composition is applied by an applicator. In other embodiments, the composition comprises from about 0.05% w/w to about 1.5% w/w tapinarof and a carrier suitable for topical administration. In other embodiments, the composition comprises from about 0.1%, 0.5% or 1% w/w tapinarof and a carrier suitable for topical administration. In other embodiments, the tapinarof is encapsulated or non-encapsulated.

In some embodiments, provided herein a method of repelling, killing or inhibiting the growth of Demodex mites in or around the eye of a subject in need thereof comprising administering a topical pharmaceutical composition comprising from about 0.01% w/w to about 2% w/w tapinarof and a carrier suitable for topical administration. In other embodiments, the method comprises topically applying the pharmaceutical composition to the cutaneous tissue surrounding the eyelash or eyelid and/or to the eyelash or the eyelid, or to the eyelid margin. In other embodiments, the Demodex mites comprise Demodex brevis or Demodex folliculorum. On other embodiments, the composition is formulated as an ointment, suspension, solution, drops, cream, lotion, gel, aerosol, transdermal patch, spray or foam. In other embodiments the composition is applied by an applicator. In other embodiments, the composition comprises from about 0.05% w/w to about 1.5% w/w tapinarof and a carrier suitable for topical administration. In other embodiments, the composition comprises from about 0.1%, 0.5% or 1% w/w tapinarof and a carrier suitable for topical administration. In other embodiments, the tapinarof is encapsulated or non-encapsulated.

In some other embodiments, there is provided a regimen of administration comprising the once daily or twice daily administration to the eyes of a patient in need thereof of a therapeutically effective amount of a topical ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration until remission or alleviation of the ocular inflammatory disease and/or the ocular degeneration disease symptoms.

In some other embodiments, there is provided a regimen of administration comprising the once daily, twice weekly, once weekly, twice monthly, monthly or every four to six weeks administration to the eyes of a patient in need thereof of a therapeutically effective amount of an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injection until remission or alleviation of the ocular inflammatory disease and/or the ocular degeneration disease symptoms.

According to some embodiments, there is provided a regimen of administration comprising the once daily or twice daily administration to the eyes of a patient in need thereof of a therapeutically effective amount of a topical ophthalmic composition for the treatment, prevention and/or amelioration of an ocular inflammatory disease and/or an ocular degeneration disease, comprising from about 0.01% w/w to about 10.0% w/w tapinarof and a carrier suitable for topical ophthalmic administration and from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, until remission or alleviation of the ocular inflammatory disease and/or the ocular degeneration disease symptoms. In another embodiment, the at least one additional active agent is selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

According to some embodiments, there is provided a regimen of administration comprising administering once daily, twice weekly, once weekly, twice monthly, monthly, every four to six weeks administration to the eyes of a patient in need thereof a therapeutically effective amount of an intraocular injectable composition for the treatment, prevention and/or amelioration of an ocular inflammatory disease and/or an ocular degeneration disease, comprising from about 3 mg/ml to about 80 mg/ml tapinarof and a carrier suitable for intraocular injectable administration and from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, until remission or alleviation of the ocular inflammatory disease and/or the ocular degeneration disease symptoms. In another embodiment, the at least one additional active agent is selected from loteprednol etabonate, prednisolone acetate, triamcinolone acetate, cyclosporin, lifitegrast, atropine, homatropine, ranibizumab, aflibercept, pegaptanib, nepafenac, meloxicam, diclofenac, bendazac, ketorolac, oxyphenbutazone, bromfenac, flurbiprofen, pranoprofen, suprofen, indomethacin, celecoxib, rofecoxib, valdecoxib, parecoxib, etoricoxib, nimesulide, etodolac, nabumetone, adalimumab, chlorambucil, cyclophosphamide, methotrexate, azathioprine, levofloxacin, gatifloxacin, moxifloxacin, ofloxacin and combinations thereof.

According to some other embodiments, there is provided a regimen of administration, comprising the once daily or twice daily administration to the eyes of a subject in need thereof a topical ophthalmic composition comprising from about 0.01% w/w to about 10.0% w/w tapinarof and a carrier suitable for topical ophthalmic administration and optionally from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), orbital myositis, and combinations thereof. According to some other embodiments, there is provided a regimen of administration, comprising the once daily, twice weekly, once weekly, twice monthly, monthly, every four to six weeks administration to the eyes of a subject in need thereof an intraocular injectable composition comprising from about 3 mg/ml to about 80 mg/ml tapinarof and a carrier suitable for intraocular injection and optionally from about 3 mg/ml to about 80 mg/ml at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), orbital myositis, and combinations thereof. In another embodiment, the ocular inflammatory disease is blepharitis.

In other embodiments, blepharitis is anterior inflammatory blepharitis. In other embodiments, blepharitis is posterior inflammatory blepharitis. In some other embodiments, blepharitis is with involvement of Demodex mites. In some other embodiments, blepharitis is without involvement of Demodex mites.

In some embodiments, provided herein regimen for treating, preventing or ameliorating blepharitis comprising administering to the eye once daily or twice daily a therapeutically effective amount of a topical pharmaceutical composition comprising from about 0.01% w/w to about 2% w/w at least one acaricidal active agent and a carrier suitable for topical administration until remission or alleviation of the blepharitis. In other embodiments, the acaricidal active agent is selected from the group consisting of ivermectin, permethrin, niclosamide, benzoyl peroxide, tapinarof and combinations thereof; wherein at least one of the acaricidal active agent is tapinarof. In other embodiments, the at least one acaricidal active agent is tapinarof. In other embodiments, the regimen comprises topically applying the pharmaceutical composition to the cutaneous tissue surrounding the eyelash or eyelid and/or to the eyelash or the eyelid, or to the eyelid margin.

In some embodiments, provided herein regimen for treating, preventing or ameliorating blepharitis comprising administering to the eye once daily or twice daily a therapeutically effective amount of a topical pharmaceutical composition comprising from about 0.01% w/w to about 2% w/w tapinarof and a carrier suitable for topical administration until remission or alleviation of the blepharitis. In other embodiments, the at least one acaricidal active agent is tapinarof. In other embodiments, the regimen comprises topically applying the pharmaceutical composition to the cutaneous tissue surrounding the eyelash or eyelid and/or to the eyelash or the eyelid, or to the eyelid margin.

In some embodiments, provided herein a regimen for repelling, killing or inhibiting the growth of Demodex mites in or around the eye of a subject once daily or twice daily a therapeutically effective amount of a topical pharmaceutical composition comprising from about 0.01% w/w to about 2% w/w at least one acaricidal active agent and a carrier suitable for topical administration until the Demodex mites are repelled, killed, or inhibited. In other embodiments, the—acaricidal active agent is selected from the group consisting of ivermectin, permethrin, niclosamide, benzoyl peroxide, tapinarof and combinations thereof. In other embodiments, the at least one acaricidal active agent is tapinarof. In other embodiments, the regimen comprises topically applying the pharmaceutical composition to the cutaneous tissue surrounding the eyelash or eyelid and/or to the eyelash or the eyelid, or to the eyelid margin.

In some embodiments, provided herein a regimen for repelling, killing or inhibiting the growth of Demodex mites in or around the eye of a subject once daily or twice daily a therapeutically effective amount of a topical pharmaceutical composition comprising from about 0.01% w/w to about 2% w/w tapinarof and a carrier suitable for topical administration until the Demodex mites are repelled, killed, or inhibited. In other embodiments, the regimen comprises topically applying the pharmaceutical composition to the cutaneous tissue surrounding the eyelash or eyelid and/or to the eyelash or the eyelid, or to the eyelid margin.

In some embodiments, there is provided a kit comprising instructions for use and one or more dosage forms of this disclosure, comprising from about 0.01% w/w to about 10.0% w/w tapinarof and a carrier suitable for topical ophthalmic administration and optionally from about 0.01% w/to about 10% w/w at least one additional active agent selected from a weak corticosteroid, an immune suppressant, an immune mediator, an antimuscarinic agent, a VGF inhibitor, an NSAID, a cytotoxic drug, a corticosteroid-sparing immunosuppressant, a TNF-α inhibitor, an antibiotic and combinations thereof, wherein the composition is formulated as an ophthalmic ointment, suspension, solution, drops, cream or foam.

In some embodiments, the compositions, kits, implants and method are for the treatment, prevention and/or alleviation of an ocular inflammatory disease and/or an ocular degeneration disease. In one embodiment, the ocular inflammatory disease and/or the ocular degeneration disease is selected from uveitis, vitritis, dry eye disease (DED), macular degeneration, idiopathic orbital inflammatory disease (IOD), chorioretinal inflammation, keratitis, blepharitis, seborrheic dermatitis of eyelids, seborrheic dermatitis of eyebrows, hyperemia, thyroid eye disease (TED), orbital myositis, age-related macular degeneration (AMD) and combinations thereof. In another embodiment the ocular inflammatory disease and/or the ocular degeneration disease is uveitis. In another embodiment the ocular inflammatory disease and/or the ocular degeneration disease is vitritis. In another embodiment the ocular inflammatory disease and/or the ocular degeneration disease is dry eye disease (DED). In another embodiment the ocular inflammatory disease and/or the ocular degeneration disease is macular degeneration. In another embodiment the ocular inflammatory disease and/or the ocular degeneration disease is idiopathic orbital inflammatory disease (IOD). In another embodiment the ocular inflammatory disease and/or the ocular degeneration disease is blepharitis. In another embodiment the ocular inflammatory disease and/or the ocular degeneration disease is seborrheic dermatitis of eyelids. In another embodiment the ocular inflammatory disease and/or the ocular degeneration disease is seborrheic dermatitis of eyebrows. In another embodiment the ocular inflammatory disease and/or the ocular degeneration disease is hyperemia. In another embodiment the ocular inflammatory disease and/or the ocular degeneration disease is thyroid eye disease (TED), In another embodiment the ocular inflammatory disease and/or the ocular degeneration disease is chorioretinal inflammation. In another embodiment the ocular inflammatory disease and/or the ocular degeneration disease is keratitis. In another embodiment the ocular inflammatory disease and/or the ocular degeneration disease is age-related macular degeneration (AMD). In another embodiment, the ocular inflammatory disease and/or the ocular degeneration disease is orbital (ocular) myositis.

In some embodiments, provided herein a kit for treating, preventing or ameliorating blepharitis or symptoms thereof comprising a topical pharmaceutical composition comprising from about 0.01% w/w to about 2% w/w at least one acaricidal active agent and a carrier suitable for topical administration wherein the composition is formulated in a dosage form selected from an ointment, suspension, solution, drops, cream, lotion, gel, aerosol, transdermal patch, spray, or foam. In other embodiments, the—acaricidal active agent is selected from the group consisting of ivermectin, permethrin, niclosamide, benzoyl peroxide, tapinarof and combinations thereof; wherein at least one of the acaricidal active agent is tapinarof. In other embodiments, the at least one acaricidal active agent is tapinarof.

In some embodiments, provided herein a kit for treating, preventing or ameliorating blepharitis or symptoms thereof comprising a topical pharmaceutical composition comprising from about 0.01% w/w to about 2% w/w tapinarof and a carrier suitable for topical administration wherein the composition is formulated in a dosage form selected from an ointment, suspension, solution, drops, cream, lotion, gel, aerosol, transdermal patch, spray, or foam.

In some embodiments, provided herein a kit for repelling, killing or inhibiting the growth of Demodex mites in or around the eye of a subject comprising a topical pharmaceutical composition comprising from about 0.01% w/w to about 2% w/w at least one acaricidal active agent and a carrier suitable for topical administration wherein the composition is formulated in a dosage form selected from an ointment, suspension, solution, cream, lotion, gel, aerosol, transdermal patch, spray or foam. In other embodiments, the—acaricidal active agent is selected from the group consisting of ivermectin, permethrin, niclosamide, benzoyl peroxide, tapinarof and combinations thereof; wherein at least one of the acaricidal active agent is tapinarof. In other embodiments, the at least one acaricidal active agent is tapinarof.

In some embodiments, provided herein a kit for repelling, killing or inhibiting the growth of Demodex mites in or around the eye of a subject comprising a topical pharmaceutical composition comprising from about 0.01% w/w to about 2% w/w a tapinarof and a carrier suitable for topical administration wherein the composition is formulated in a dosage form selected from an ointment, suspension, solution, cream, lotion, gel, aerosol, transdermal patch, spray or foam.

### Definitions

Unless defined otherwise, all technical and scientific terms used herein have the same meaning as is commonly understood by one of skill in the art to which the invention pertains. In case of conflict, the specification, including definitions, takes precedence. All patents, patent applications, published applications, articles, publications and other published materials referred to throughout the entire disclosure herein, unless noted otherwise, are incorporated by reference in their entirety.

As used herein, the indefinite articles “a” and “an” mean “at least one” or “one or more” unless the context clearly dictates otherwise.

As used herein, the term “treating” or “treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition.

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

The term “ingredient” refers to a pharmaceutically acceptable ingredient which is included or is amenable to be included in FDA's Inactive Ingredient database (IIG). Inactive ingredients sometimes exhibit some therapeutic effects, although they are not drugs.

As used herein, a “pharmaceutical composition” refers to a composition comprising one or more active ingredients with other components such as pharmaceutically acceptable ingredients or excipients. The purpose of a pharmaceutical composition is to facilitate administration of an active ingredient to a subject.

As used herein, the term “micelle” or “nanomicelle” refer to an aggregate (or cluster) of surfactant molecules. In some embodiments, ophthalmic compositions of the present invention include an aqueous, clear or mixed micellar solution. Addition of water to a drug polymer mixture in organic solvent should spontaneously generate micelles thereby entrapping the pharmaceutical active agent in the hydrophobic core of mixed nanomicelles.

The term “Intraocular injection” refers herein to an injection into the eye. Non limiting examples include injection into the vitreous (intravitreous injection) or subretinal (interphotoreceptor) space; into the tissue surrounding the eye, or intraocular injection refers to periocular and periorbital injection.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges by the term “about” should not be considered to be limited to the recited range. Rather, numerical ranges preceded by the term “about” should be understood to include a range accepted by those skilled in the art for any given element in formulations according to the present invention.

As used herein, when a numerical value is preceded by the term “about”, the term “about” is intended to indicate +/−10%.

The terms “comprise”, “comprising”, “includes”, “including”, “having” and their conjugates mean “including but not limited to”.

The term “consisting of” means “including and limited to”.

The term “consisting essentially of” means that the composition, method formulation may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include chemical, molecular and biochemical, techniques. Such techniques are thoroughly explained in the literature. General references are provided throughout this document. The procedures therein are believed to be well known in the art and are provided for the convenience of the reader. All the information contained therein is incorporated herein by reference.

### Example 1

### Preparation of a Tapinarof Ophthalmic Ointment Composition

The topical ophthalmic tapinarof ointment consists of:
0.01-10.0% w/w tapinarof,
50-65% w/w white petrolatum,
6% woolfat,
15-30% w/w liquid paraffin,
3-10% w/w PEG-4000 or PEG-400
0.5% phenylethyl alcohol
0.1-1.0% w/w alpha-tocopherol

The ointment composition is prepared by the following steps:
1. Weigh liquid paraffin in a bottle. Remove air bubbles in the liquid paraffin by applying vacuum for a period of 30 minutes.
2. Melt white petrolatum and wool fat on a stir plate at approximately 70° C.
3. Mix gently the liquid paraffin, white petrolatum and wool fat at approximately 70° C. for a period of 15 minutes or until the ingredients are uniformly mixed.
4. Weigh tapinarof having an average particle size of less than 1 μm and (partly) dissolve it in PEG 400 or PEG 4000 by sonicating in a water bath maintained at 45° C.
5. Add alpha-tocopherol and phenylethyl alcohol to the PEG containing tapinarof.
6. Add (partly) dissolved tapinarof and mix for a period of 45 minutes or until a uniform dispersion is obtained.
7. Sterilize the tapinarof topical ophthalmic ointment by filtering the molten ointment through a 0.2-micron filter.
8. Fill the sterilized tapinarof ointment in a tube or other delivery system.

### Example 2

Preparation of a Tapinarof Eye-Drop Suspension Composition

The topical eye drop tapinarof suspension composition consists of:
0.01-10.0% w/w tapinarof,
0.1-0.3% w/w edetate disodium,
0.2-1.0% w/w glycerin,
0.1-1% w/w povidone,
0.1-0.25% w/w tyloxapol,
0.01% benzalkonium chloride.

Add hydrochloric acid or sodium hydroxide to adjust the pH to 5.3-5.6.

The suspension is sterile and essentially isotonic.

### Example 3

Preparation of a Tapinarof/Prednisolone Acetate Eye-Drop Suspension Composition

The topical eye drop tapinarof/prednisolone acetate suspension composition consists of:
0.01-10.0% w/w tapinarof,
1% w/w prednisolone acetate,
0.1-0.3% w/w edetate disodium,
0.2-1.0% w/w glycerin,
0.1-1% w/w povidone,
0.1-0.25% w/w tyloxapol,
0.01% benzalkonium chloride.

Add hydrochloric acid or sodium hydroxide to adjust the pH to 5.3-5.6.

The suspension is sterile and essentially isotonic.

### Example 4

In Vitro Activity of Tapinarof Based Formulations in Killing Demodex folliculorum

The purpose of this study is to evaluate the effectiveness of a composition comprising Tapinarof in killing Demodex for clinical treatment.

Subjects included in this study exhibited clinical evidence of Demodex blepharitis (cylindrical dandruff) confirmed by light microscopy. Subjects were excluded if they were less than 18 years of age, lacked the ability to consent, or displayed signs of an active ocular infection. Following informed consent, one to four lashes with significant cylindrical dandruff were selected for epilation. Lashes were firmly grasped near the base and gently teased out with a slow circular motion to prevent cylindrical dandruff and Demodex from being left behind on the lid margin.

Epilated lashes were then placed onto a glass slide and the number of live organisms under light microscopy was then recorded. Lashes with live Demodex were then included in the study. Each lash obtained was then assigned to one of 13 study reagents (Table 1).

These reagents included 1) 50% transcutol, 2) 50% acetone, 3) 50% acetonitrile, 4) 50% ethanol, 5) 50% isopropyl alcohol, 6) 80% transcutol, 7) 80% acetone, 8) 80% acetonitrile and 9-11) tapinarof concentrations (50 mg/0.1%, 250 mg/0.5% and 500 mg/1%) with 50% isopropyl alcohol. Positive and negative controls were also evaluated as separate solutions of 12) 50% tea tree oil/50% macadamian nut oil and 13) 100% basic saline solution respectively.

Observation of the study material with the Demodex took place over 90 minutes with checks under light microscopy every five minutes for cessation of Demodex activity. Organism death was recorded when all rotary movement of Demodex appendages ceased. At least five lashes with live Demodex were evaluated for each reagent. Results were recorded in Microsoft Excel and statistical analysis for significant differences in average time to death were done with two-tailed t-test using the same software (Table 1). The value 90 was used to denote no effect. P-values less than 0.05 were considered significant.

TABLE 1
Study results of eye lashes with live Demodex

| | | | | Average | |
|---|---|---|---|---|---|
| | | Number of | | Time to | |
| | | Demodex | Kill | Death | P- |
| | Test Reagents | Tested | Rate | (minutes) | value |
| 1 | Transcutol 50% | 8 | 100% | 35 | <.005 |
| 2 | Acetonitrile 50% | 8 | 88% | 31.43 | <.005 |
| 3 | Acetone 50% | 12 | 33% | 82.5 | 0.05 |
| 4 | Ethanol 50% | 11 | 100% | 33.64 | <.005 |
| 5 | Isopropyl alcohol 50% | 10 | 0% | >90 | 1 |
| 6 | Transcutol 80% | 18 | 100% | 15 | <.005 |
| 7 | Acetone 80% | 14 | 29% | 9.7 | 0.04 |
| 8 | Acetonitrile 80% | 6 | 100% | 5.83 | <.005 |
| 9 | Tapinarof 0.1% in 50% | 13 | 8% | 80 | .34 |
| | isopropyl alcohol | | | | |
| 10 | Tapinarof 0.5% in 50% | 14 | 29% | 10.82 | .049 |
| | isopropyl alcohol | | | | |
| 11 | Tapinarof 1% in 50% | 15 | 33% | 60 | .047 |
| | isopropyl alcohol | | | | |
| 12 | Tea tree oil 50%/ | 10 | 100% | 12.75 | <.005 |
| | Macadamia nut oil 50% | | | | |
| 13 | Balance Salt Solution 100% | 10 | 0% | >90 | |

### Results:

Live Demodex were present on 92 eyelashes that were taken from 55 participants. The positive control group of 50% tea tree oil and 50% macadamia nut oil yielded a 100% kill rate and an average time of death of 12.75 minutes (p<0.005). Tapinarof 0.1% yielded an 8% kill rate, and an average time of death 80 minutes, p=0.34. Tapinarof 0.5% yielded a 29% kill rate, and an average time of death 10.82 minutes, p=0.049. Tapinarof 1% yielded a 33% kill rate, and an average time of death 60 minutes, p=0.047.

Transcutol 50% and 80% both had 100% kill rates, and an average time of death of 35 and 15 minutes respectively. (p<0.005).

Acetonitrile 50% and 80% demonstrated kill rates of 88% and 100%, and an average time of death of 31.43 and 5.83 minutes respectively (p<0.005).

Acetone 50% and 80% demonstrate 39% and 29% kill rates, and an average time of death of 82.5 and 9.7 minutes respectively (p=0.05 and 0.04 respectively).

Isopropyl alcohol demonstrated a 0% kill rate, and was statistically equivalent to the control.

This study examined the potential Democidal effects of tapinarof. The results showed a moderate in vitro killing effect for tapinarof against Demodex mites at the 0.5% and 1% concentrations. This effect is dose dependent and a significant effect was not observed at the 0.1% concentration (p=0.34). However, at concentrations greater than 0.5%, there was not a statistically significant change in the killing effect of tapinarof (p=0.49). This efficacy is lower than that of the positive control, 50% tea tree oil, used in our study. Note that tea tree oil at concentrations of 5% to 50% are typically used clinically, but it has been noted that strong converging evidence for its effectiveness at treating and improving the ocular surface symptoms in those with Demodicosis is still lacking.

Various excipients and or solvents used in the pharmaceutical industry were examined for democidal potential in this study including: transcutol, acetone, acetonitrile, ethanol, and isopropyl alcohol. Transcutol is an excipient that has become increasingly used in cosmetics and oral, topical, transdermal, and injectable pharmaceutical compounds.

Transcutol has been shown to enhance drug solubilization, percutaneous absorption rate, drug retention in the skin, however, studies have brought its nephrotoxicity into question.

Isopropyl alcohol is additionally employed in the pharmaceutical industry, and when ingested in great quantities can result in CNS and respiratory depression.

Acetone is used frequently in the pharmaceutical industry and is rarely a cause of poisoning, however, it's toxicity can be increased by concurrent ethanol exposure.

Ethanol has many uses in medicine as both an embolic, ablative, and sclerosing agent.

Acetonitrile is banned in European cosmetics secondary to its association with cyanide poisoning.

What is claimed is:

1. A method of treating, preventing or ameliorating blepharitis or symptoms thereof, comprising administering a topical pharmaceutical composition comprising from about 0.01% w/w to about 2% w/w tapinarof and a carrier suitable for topical administration.

2. The method of claim 1, wherein the method comprises topically applying the pharmaceutical composition to the cutaneous tissue surrounding the eyelash or eyelid and/or to the eyelash or the eyelid, or to the eyelid margin.

3. The method of claim 1, wherein the blepharitis is selected from anterior inflammatory blepharitis or posterior inflammatory blepharitis.

4. The method of claim 1, wherein the method further repels, kills or inhibits the growth of Demodex mites in or around the eye.

5. The method of claim 1, wherein the blepharitis is with involvement of Demodex mites.

6. The method of claim 4, wherein the Demodex mites comprise Demodex brevis or Demodex folliculorum.

7. The method of claim 5, wherein the Demodex mites comprise Demodex brevis or Demodex folliculorum.

8. The method of claim 1, wherein the composition is formulated as an ointment, suspension, spray, solution, drops, cream, lotion, gel, aerosol, transdermal patch, drops, or foam.

9. The method of claim 8, wherein the composition is applied by an applicator.

10. The method of claim 1, wherein the composition comprises from about 0.05% w/w to about 1.5% w/w tapinarof and a carrier suitable for topical administration.

11. The method of claim 1, wherein the composition comprises from about 0.1%, 0.5% or 1% w/w tapinarof and a carrier suitable for topical administration.

12. The method of claim 1, wherein said blepharitis symptoms are selected from redness, irritation, burning, itching, tearing, crusting of eyelashes, and madarosis.

13. A regimen for treating, preventing or ameliorating blepharitis comprising administering to the eye once daily or twice daily a therapeutically effective amount of a topical pharmaceutical composition comprising from about 0.01% w/w to about 2% w/w tapinarof and a carrier suitable for topical administration until remission or alleviation of the blepharitis.

14. The regimen of claim 13, wherein the regimen comprises topically applying the pharmaceutical composition to the cutaneous tissue surrounding the eyelash or eyelid and/or to the eyelash or the eyelid, or to the eyelid margin.

15. A method of repelling, killing or inhibiting the growth of Demodex mites in or around the eye of a subject in need thereof comprising administering a topical pharmaceutical composition comprising from about 0.01% w/w to about 2% w/w tapinarof and a carrier suitable for topical administration.

16. The method of claim 15, wherein the method comprises topically applying the pharmaceutical composition to the cutaneous tissue surrounding the eyelash or eyelid and/or to the eyelash or the eyelid, or to the eyelid margin.

17. The method of claim 15, wherein the Demodex mites comprise Demodex brevis or Demodex folliculorum.

18. The method of claim 15, wherein the composition is formulated as an ointment, suspension, spray, solution, cream, lotion, gel, aerosol, transdermal patch, drops, or foam.

19. The method of claim 18, wherein the composition is applied by an applicator.

20. The method of claim 15, wherein the composition comprises from about 0.05% w/w to about 1.5% w/w tapinarof and a carrier suitable for topical administration.

21. The method of claim 15, wherein the composition comprises from about 0.1%, 0.5% or 1% w/w tapinarof and a carrier suitable for topical administration.

22. A regimen for repelling, killing or inhibiting the growth of Demodex mites in or around the eye of a subject once daily or twice daily a therapeutically effective amount of a topical pharmaceutical composition comprising tapinarof and a carrier suitable for topical administration until the Demodex mites are repelled, killed, or inhibited.

23. The regimen of claim 23, wherein the regimen comprises topically applying the pharmaceutical composition to the cutaneous tissue surrounding the eyelash or eyelid and/or to the eyelash or the eyelid, or to the eyelid margin.
